(19) United States
(12) Patent Application Publication
(10) Pub. No.: US 2020/0317548 A1
(43) Pub. Date: Oct. 8, 2020

(54) SYSTEM FOR THE DELIVERY OF TRANSDERMAL NUTRIENTS AND GASSES

(71) Applicants: Leo Szymborski, Palm City, FL (US); Aida Aloian, Palm City, FL (US)

(72) Inventors: Leo Szymborski, Palm City, FL (US); Aida Aloian, Palm City, FL (US)

(73) Assignee: Derma Shower, LLC., Palm City, FL (US)

(21) Appl. No.: 16/375,252

(22) Filed: Apr. 4, 2019

(51) Int. Cl. C02F 1/68; C02F 1/00; B01D 29/11; A61K 9/00; A61K 9/08

(52) U.S. Cl. CPC C02F 1/68; C02F 1/003; C02F 2101/322; A61K 9/0014; A61K 9/08; B01D 29/11

(57) ABSTRACT

A water filter and nutrient delivery system comprising a water inlet in fluid communication with a water supply via a conduit is provided. The water inlet is configured to transmit a flow of water to a filter system comprised of a filter media to remove contaminants from the water flow and expel filtered water. An additive system receives the expelled water from the filter system and is comprised of an additive capsule containing an additive composition to treat the water flow. A mixer combines the additive composition into the water flow and expels to treated water through a water outlet.

### TECHNICAL FIELD

The embodiments generally relate to a system and method for the delivery of transdermal nutrients and gasses to a human, and more specifically, relate to shower faucets which filter incoming water and supplement outgoing water with transdermally absorbable nutrient and gasses.

### BACKGROUND

Residents often rely on local governments to supply water to the residence, the quality of which varies by region and municipality. There are thousands of parameters for determining water quality, including microbiological and physiochemical factors. In order to disinfect a water supply, many providers use varying degrees of chlorination, ultraviolet light, or other known forms of water treatment. Once treated, the water is transmitted to residences using various means.

Public water may also be treated domestically by the resident to remove undesired constituents. Common techniques such as reverse osmosis and distillation require high energy input and are at times impractical.

Humans have the ability to absorb certain compounds via the transdermal route. This technique is used in drug delivery when there is a significant first-pass effect of the liver which can prematurely metabolize some compounds. In such, many nutrients and gasses can be provided in water and subsequently absorbed by the body. Many of these compounds are required by the body for the proper function of various biological processes. Some estimates determine that 90% of Americans live with a nutrient imbalance, leaving dangerous free radicals present in the body.

In the current arts, the domestic water filtration system can be expensive and impractical to install. Further, these systems not only remove the potentially harmful contaminants, but also remove beneficial minerals in the water. While these minerals may be replaced by dietary additions, bath tablets, or similar implements, these protocols often result in reduced adherence due to the inherent inconvenience of supplementing these activities into a daily routine.

### SUMMARY OF THE INVENTION

This summary is provided to introduce a variety of concepts in a simplified form that is further disclosed in the detailed description of the invention. This summary is not intended to identify key or essential inventive concepts of the claimed subject matter, nor is it intended for determining the scope of the claimed subject matter.

A water filter and nutrient delivery system is disclosed comprising a water inlet in fluid communication with a water supply via a conduit. The water inlet is configured to transmit a flow of water to a filter system comprised of a filter media to remove contaminants from the water flow and expel filtered water. An additive system receives the expelled water from the filter system and is comprised of an additive capsule containing an additive composition to treat the water flow. A mixer combines the additive composition into the water flow and expels the treated water through a water outlet.

The water filter and nutrient delivery system provides an on-demand water filter and additive system to a shower faucet, allowing the user to select from a plurality of nutrients which are transdermally absorbed while showering.

In one aspect, the additive composition is a plurality of solids and a plurality of gasses.

In one aspect, the additive composition is transdermally absorbed by a human.

In one aspect, the additive composition is provided in a single-use capsule, wherein the single-use capsule is punctured by an opener provided in an additive system housing.

In one aspect, the additive composition is selectable by a user via an additive selection interface provided on the additive system housing.

In one aspect, a plurality of magnets are disposed at the perimeter of the water inlet to structure the water flow.

In one aspect, the housing of the mixer is dimensioned to provide a suitable pressure to dissolve one or more gasses into the filtered water.

In one aspect, the additive system is further comprised of an upper assembly having an interface including the water inlet and the water outlet.

In one aspect, the transdermally absorbable additive composition is selectable by a user.

In one aspect, the single-use capsule is punctured by an opener provided in an additive system housing to release the transdermally absorbable additive composition into the water flow.

In one aspect, the additive system is further comprised of an opening in fluid communication with the water inlet. The opening is downstream of the filter media to expel the filtered water to the additive capsule.

In one aspect, the additive system receives the water flow via a valve having a valve knob to selectively control the water flow. The valve is comprised of an inlet to deliver the water flow into the additive capsule containing the transdermally absorbable additive composition. The water flow is transmitted through a bottom end of the additive capsule and into the mixer via one or more channels.

### BRIEF DESCRIPTION OF THE DRAWINGS

A complete understanding of the present invention and the advantages and features thereof will be more readily understood by reference to the following detailed description when considered in conjunction with the accompanying drawings wherein:

FIG. 1 illustrates a perspective view of the filter and water additive system, according to some embodiments;

FIG. 2 illustrates an exploded view of the upper assembly of the filter and water additive system components, according to some embodiments;

FIG. 3 illustrates an exploded view of the assembly of the filter system and water additive system components, according to some embodiments;

FIG. 4A illustrates a cross-sectional view of the water filter and additive assembly, according to some embodiments;

FIG. 4B illustrates a cross-sectional view of the water filter and additive assembly, according to some embodiments;

FIG. 5 illustrates a perspective view of the additive system, according to some embodiments;

FIG. 6 illustrates an exploded view of the additive system components, according to some embodiments;

FIG. 7 illustrates a cross-sectional view of the additive system and the flow of water through the additive capsule, according to some embodiments;

FIG. 8 illustrates a perspective view of the additive capsule, according to some embodiments;

FIG. 9 illustrates a perspective view of the water structuring system, according to some embodiments; and

FIG. 10 illustrates an exploded view of the water structuring system, according to some embodiments.

### DETAILED DESCRIPTION

The specific details of the single embodiment or variety of embodiments described herein are to the described system and methods of use. Any specific details of the embodiments are used for demonstration purposes only and not unnecessary limitations or inferences are to be understood therefrom.

Before describing in detail exemplary embodiments, it is noted that the embodiments reside primarily in combinations of components and procedures related to the system and method. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second” and the like, may be used solely to distinguish one entity or element from another entity or element without necessarily requiring or implying any physical or logical relationship or order between such entities or elements.

In general, the embodiments presented herein relate to a shower faucet having a filter system to remove volatile organic chemicals (VOC's) from water supply. The faucet also includes an additive system which permits the introduction of an additive composition comprising nutrients and gasses into the filtered water to be delivered transdermally to the user while bathing. The additive composition may include liquid or dissolved particles introduced into a water flow.

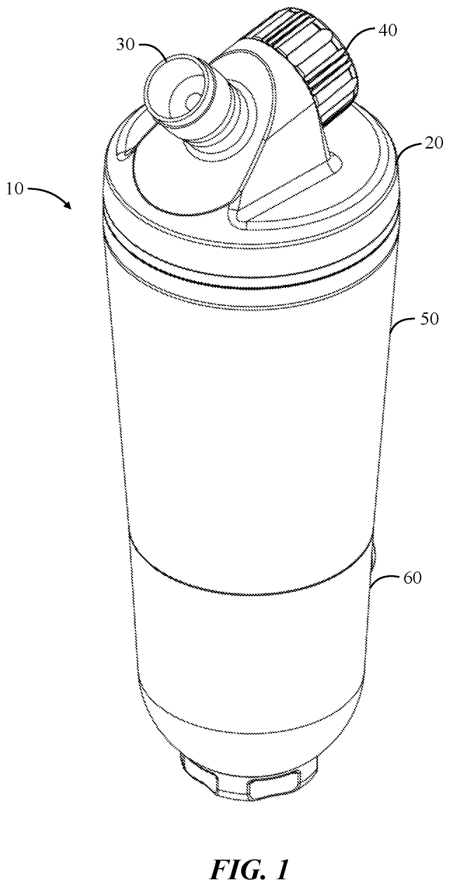

FIG. 1 illustrates the shower faucet filter and additive system 10 (hereinafter referred to as the “system”) in an exemplary embodiment. The system 10 is comprised of an upper assembly 20 providing an interface for a water inlet 30 and a water outlet 40. The water inlet 30 engages with a water supply conduit to receive water from a water supply and transmits the ingressing water to a filter system 50 to remove unwanted particulates, contaminants, chemicals, or the like from the ingressing water to produce filtered water through the water outlet 40 adapted to engage with a shower faucet or similar water outlet. The system 10 is further comprised of a water additive system 60 which allows the user to selectively provide nutrient additives to the water supply.

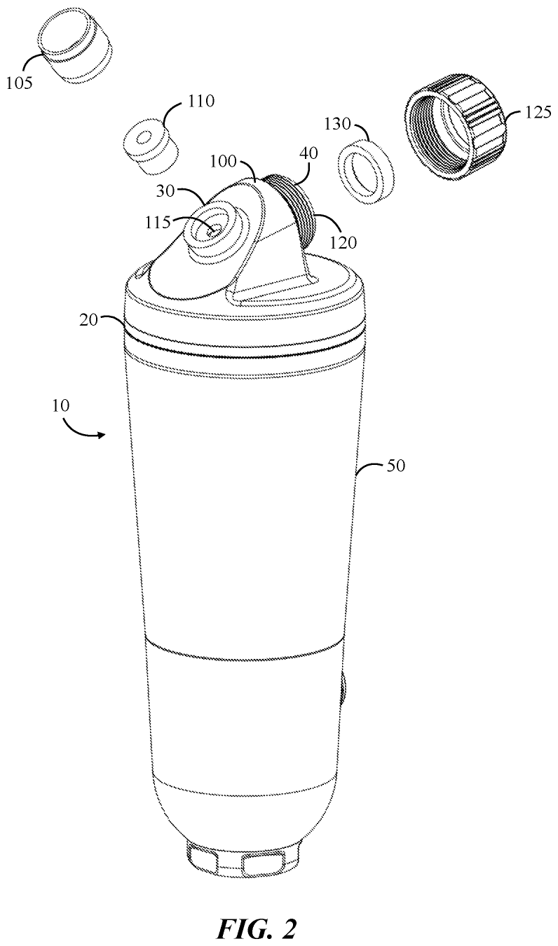

FIG. 2 illustrates an exploded view of the upper assembly 20 of the system. The upper assembly includes an interface 100 having the water inlet 30 and water outlet 40. The water inlet is comprised of a collar 105 positioned over an inlet component 110 configured to transmit water to the filter system 50 via an aperture 115. A water outlet 40 is positioned opposite the water inlet 30. The water outlet is comprised of a male threaded coupling 120 and female threaded coupling 125 having a gasket 130 disposed of therebetween. The water outlet 40 receives filtered water, which may be transmitted to the additive system 60, and dispenses the treated water to the shower faucet.

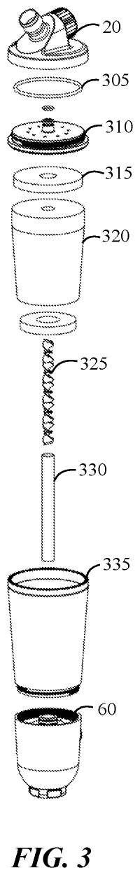

FIG. 3 illustrates an exploded view of the system 10 components. The upper assembly 20, filter system 50, and additive system 60 releasably engage to form a hermetically sealed housing for the internal components of the system 10. A first insert 305 is positioned between the upper assembly 20 and a filter end cap 310. A second insert 315 is positioned between the filter end cap 310 and a filter media 320 to filter contaminants from the water supply. A mixer 325 is in communication with a riser 330 within a central portion to receive water from the water inlet and transmit the water to the additive system 60 engaged with the bottom of a central portion 335 of the housing. Each component is comprised of a central aperture to facilitate the transfer of water from the water inlet to the filter 325 and additive system 60.

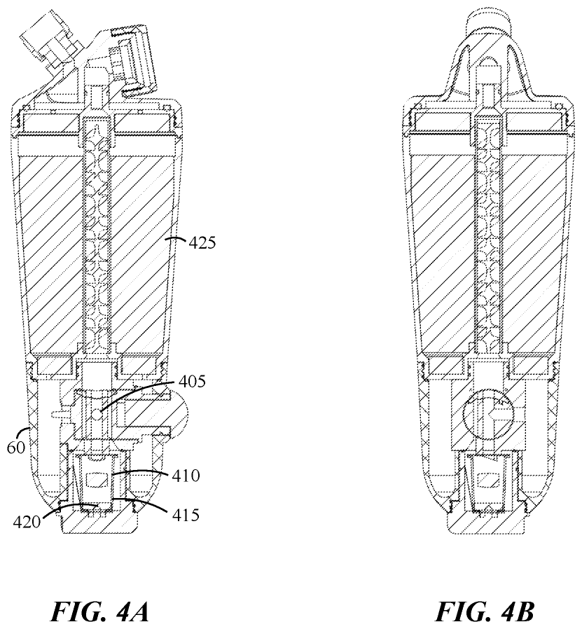

FIG. 4A and FIG. 4B illustrate cross-section views of the system 10. An opening 410 is positioned within the interior of the additive system 60 downstream of the filter media 320 and mixer 325. Water flows through the opening 405 and into the additive pod 410 having an exterior 415. The exterior 415 is punctured by an opener 420 to permit the flow of water to dissolve the additive contents. Water then flows up through the outer diameter 425 wherein the additive is further mixed into the water before egressing through the water outlet.

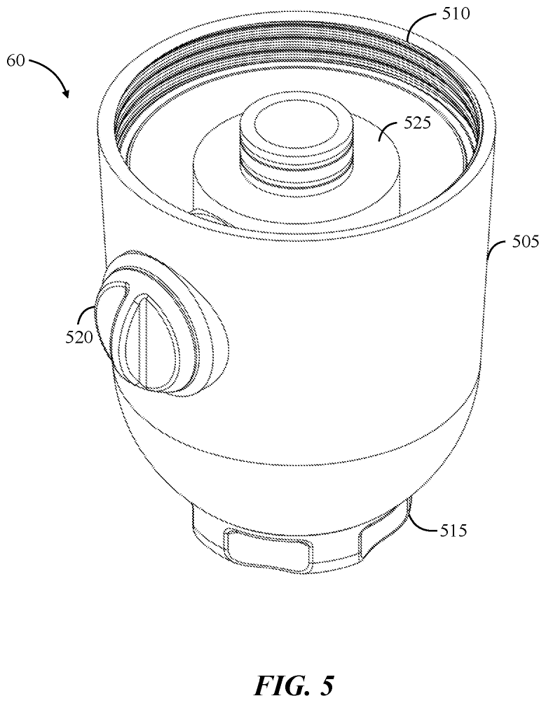

FIG. 5 illustrates the additive system 60 having a housing 505 to contain the additive composition therein. The housing 505 has a top end 510 and a bottom end 515. The top end includes an aperture to permit the ingress of water into the interior of the housing 505 to interface with the additive capsule therein. The top end of the housing 505 releasably engages with the bottom end of the central portion via a threaded engagement or similar releasable attachment means. A valve knob 520 provides an adjustment means for the water flow into and out of the valve 525 controlling the flow of water through the additive capsule.

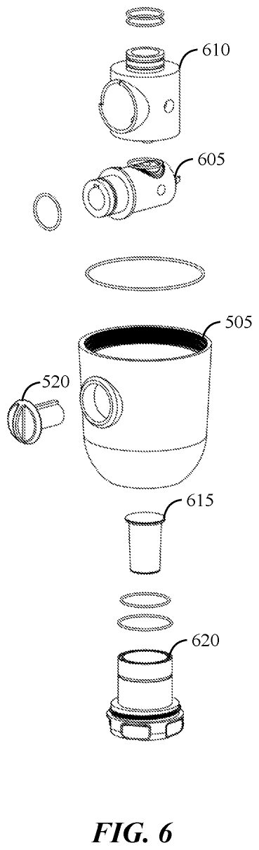

FIG. 6 illustrates an exploded view of the additive system 60 components. The additive system is comprised of a valve 605 disposed within a valve casing 610 to control the water flow through the additive capsule 615 which contains the additive composition. An additive capsule casing 620 is releasably engaged with the bottom end of the additive system housing 505 to allow the user to selectively replace the additive capsule 615 as needed. The valve knob 520 is attached to the side of the additive system housing 60 to adjust the valve 605 position and permit the user to selectively determine the water flow rate into the additive system 60. In some embodiments, the valve 605 may be adjusted to an off position via the valve knob 520 if the user does not want to add the additive composition to the water flow, or if no additive composition is provided during use.

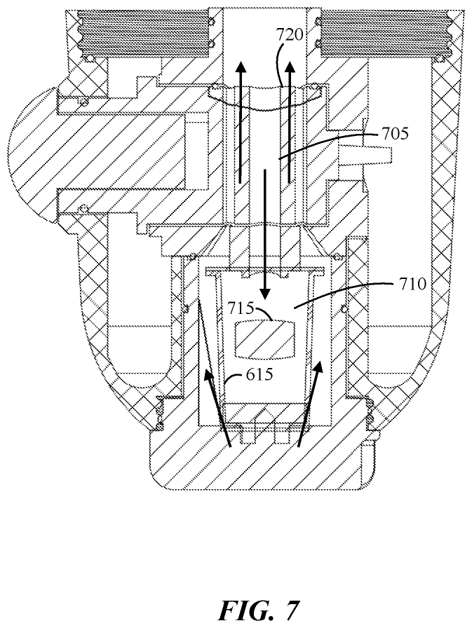

FIG. 7 illustrates a cross-sectional view of the additive system 60. The arrows illustrate the flow of water through the additive system inlet 705. Water flows into the additive system inlet 705 is controlled by the valve knob 520 in communication with the valve 605. Water flows into the interior 710 of the additive capsule 615 to interface with the additive composition 705 disposed of therein. The additive composition is at least partially dissolved in or intermixed with the water flow which flows through the bottom end of the additive capsule 615 and up to the mixer via channels 715, 720.

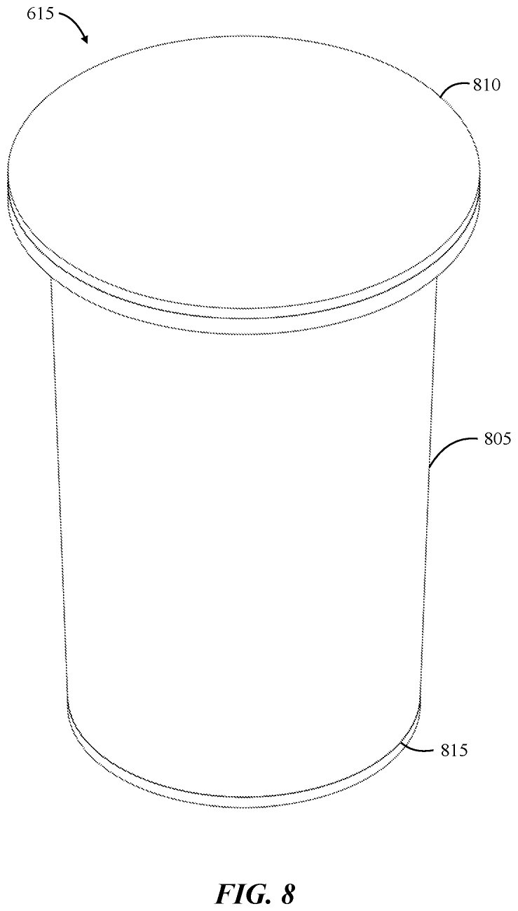

FIG. 8 illustrates the additive capsule 615 having a central portion 805 extending between a top end 810 and a bottom end 815 to define a housing for the additive composition. The additive capsule 615 is hermetically sealed to preserve the additive composition which is disposed within the additive capsule 615 at the time of manufacture. The additive capsule 615 is constructed of a material that is penetrable by the opener such that the flow of water may interface with the additive composition and effectively mix with the water flow.

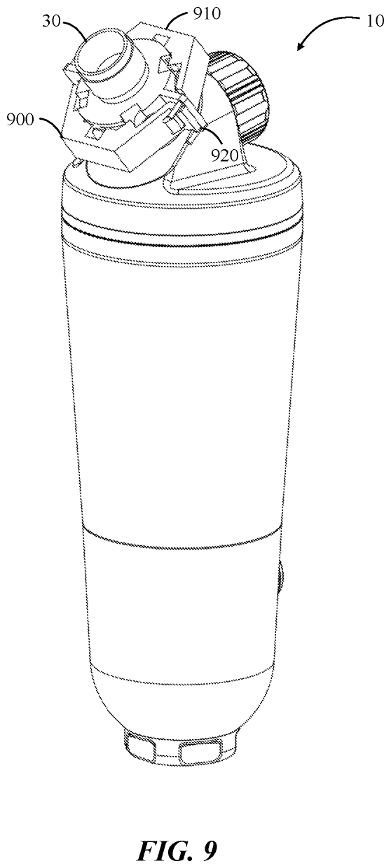

FIG. 9 illustrates an alternative embodiment wherein a water structuring assembly 900 is in communication with the water inlet 30 to structure the water ingressing into the system 10. The water structuring assembly 900 is comprised of a collar 910 circumferentially engaged with the exterior of the water inlet 30. The water structuring assembly 900 is comprised of an attachment means 920 such as a latch, locking clamp, or similar attachment means to releasably engage the collar 910 to the water inlet 30. The water structuring assembly 900 removes the need for users to install a central water structuring system in their home and provides an on-demand water structuring system for the shower faucet.

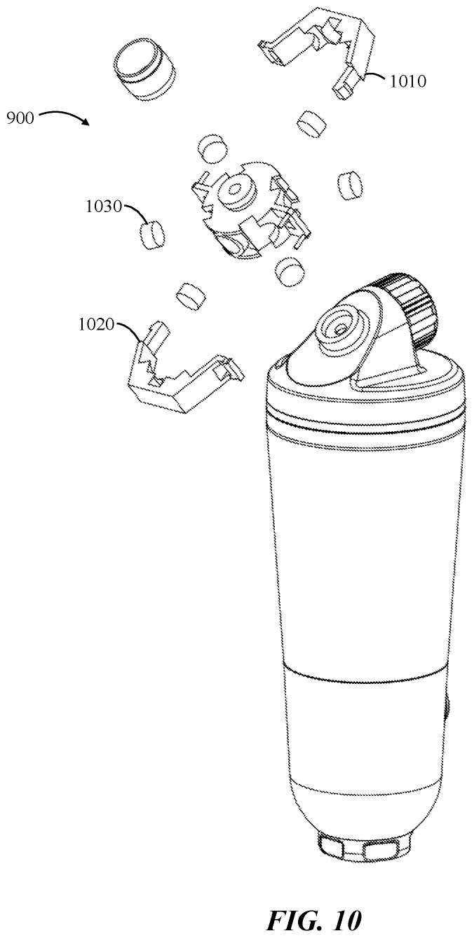

FIG. 10 illustrates an exploded view of the water structuring system 900. The collar is comprised of a first half 1010 and a second half 1020 dimensioned to releasably engage with the water inlet. A plurality of magnets 1030 is retained within the water structuring system 900 to structure the water travelling through the water inlet.

In some embodiments, the water structuring system 900 provides a means for altering the chemical configuration of the water molecules to enhance the nutrient absorption properties, remove metabolic wastes, and enhance cellular communication.

In some embodiments, the additive capsule may be a single-use capsule, wherein the user replaces the capsule by disengaging the additive capsule casing and ejecting the used additive capsule. The user may then replace the additive capsule and engage the additive capsule casing before use.

In some embodiments, the additive composition is provided as a capsule, a pod, a loose material composition, a formed material composition, or similar configuration such that the additive composition may interface with and at least partially mix with the flow of water into the additive capsule.

In some embodiments, the additive composition is at least partially comprised of a gas that may be condensed into the water flow. In one example, the water may be hydrogenated to enhance the antioxidant properties of the water. To effectively condense the gasses into the water flow, the mixer may be dimensioned to impart a suitable pressure to the water flow.

The embodiments allow for an effective means for an additive composition to be dispensed directly into the water flow to create a unique showering experience for the user. The additive composition is not limited to a single composition. Further, the system may utilize a plurality of additive compositions at once.

In some embodiments, the filter media may include a combination of components including various compounds. In one example, the filter media is comprised of kinetic degradation fluxion (KDF) 55, calcium sulfite, activated carbon, and far-infrared ceramic stones.

In some embodiments, the filter and/or the filter media may be replaced following a period when the filter media is no longer effective for removing contaminants in the water supply. To test the effectiveness of the filter and filter media, the user may be provided with a test kit, such as a chlorine test kit known in the arts. The user can determine if a threshold value of chlorine is present in the water supply and replace the filter and/or the filter media if the threshold value is reached or exceeded. The user may be provided with a testing protocol, such as testing the chlorine levels exiting the shower faucet each week, after a predetermined number of uses, or after a predetermined period of active filtration time (measured by the time water is flowing through the filter).

In some embodiments, a soap or dye may be added to the additive composition. This may make it obvious to users that they are getting the additive composition in the water.

In some embodiments, a fragrance may be added to the additive composition to enhance the user experience.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A water filter and nutrient delivery system, comprising:
a water inlet in fluid communication with a water supply via a conduit, the water inlet configured to transmit a flow of water to a filter system comprised of a filter media to remove contaminants from the water flow and expel filtered water;
an additive system to receive the expelled water from the filter system, the additive system comprised of an additive capsule containing an additive composition to treat the water flow;
a mixer to at least partially mix the additive composition into the water flow; and
a water outlet to permit the egress of filtered and treated water flow from the mixer through the water outlet.

2. The system of claim 1, wherein the additive composition is comprised of a plurality of solids and a plurality of gasses.

3. The system of claim 2, wherein the additive composition is transdermally absorbed by a human.

4. The system of claim 1, wherein the additive composition is provided in a single-use capsule, wherein the single-use capsule is punctured by an opener provided in an additive system housing.

5. The system of claim 4, wherein the additive composition is selectable by a user via an additive selection interface provided on the additive system housing.

6. The system of claim 1, further comprising a plurality of magnets disposed at the perimeter of the water inlet to structure the water flow.

7. The system of claim 1, wherein the housing of the mixer is dimensioned to provide a suitable pressure to conde one or more gasses into the filtered water.

8. The system of claim 1, further comprising an upper assembly comprised of an interface including the water inlet and the water outlet.

9. A water filter and nutrient delivery system, comprising:
a water inlet in fluid communication with a water supply via a conduit, the water inlet configured to transmit a flow of water to a filter system comprised of a filter media to remove contaminants from the water flow and expel filtered water to an additive system;
an additive capsule contained within the additive system and in fluid communication with the water inlet, the additive capsule comprised of a transdermally absorbable additive composition at least partially dissolvable into the water flow via a mixer; and
a water outlet to permit the egress of the water flow from the mixer through the water outlet to a shower faucet.

10. The system of claim 9, wherein the transdermally absorbable additive composition is a plurality of solids and a plurality of gasses.

11. The system claim 10, wherein the transdermally absorbable additive composition is selectable by a user.

12. The system of claim 11, wherein the transdermally absorbable additive composition is provided in a single-use capsule, wherein the single-use capsule is punctured by an opener provided in an additive system housing to release the transdermally absorbable additive composition into the water flow.

13. The system of claim 12, wherein the housing of the mixer is dimensioned to provide a suitable pressure to dissolve one or more gasses into the filtered water.

14. The system of claim 9, further comprising a plurality of magnets disposed at the perimeter of the water inlet to structure the water flow.

15. The system of claim 9, wherein the additive system is further comprised of an opening in fluid communication with the water inlet, wherein the opening is downstream of the filter media, and wherein the opening expels the filtered water to the additive capsule.

16. The system of claim 9, wherein the filter media is configured as a static filter.

17. The system of claim 9, further comprised of a filter indicator.

18. A water filter and nutrient delivery system, comprising:
a water inlet in fluid communication with a water supply via a conduit, the water inlet configured to transmit a flow of water to a filter system comprised of a filter media to remove contaminants from the water flow and expel filtered water to an additive system, the water inlet further comprised of a water structuring system to structure the water flow ingressing into the water inlet;
an additive capsule contained within the additive system and in fluid communication with the water inlet, the additive system receiving the water flow via a valve including a valve knob to selectively control the water flow, the additive capsule comprised of a transdermally absorbable additive composition at least partially dissolvable into the water flow via a mixer, the transdermally absorbable additive composition comprised of at least one transdermally absorbable nutrient and at least one gas; and
a water outlet to permit the egress of the water flow from the mixer through the water outlet to a shower faucet to provide the water flow to the skin of a human.

19. The system of claim 18, wherein the valve is comprised of an inlet to deliver the water flow into the additive capsule containing the transdermally absorbable additive composition, wherein the water flow is directed through a bottom end of the additive capsule and into the mixer via one or more channels.

20. The system of claim 19, wherein the mixer is dimensioned to impart a suitable pressure to effectively dissolve the transdermally absorbable additive composition into the water flow.
